(19) United States
(12) Patent Application Publication
(10) Pub. No.: US 2024/0050134 A1
(43) Pub. Date: Feb. 15, 2024

(54) INTERMEDULLARY LENGTHENING IMPLANT WITH INTEGRATED LOAD SENSOR

(71) Applicant: NuVasive Specialized Orthopedics, Inc., San Diego, CA (US)

(72) Inventors: Nathan Meyer, Vista, CA (US); Youngsam Bae, Placentia, CA (US)

(21) Appl. No.: 17/887,789

(22) Filed: Aug. 15, 2022

(51) Int. Cl. A61B 17/72

(52) U.S. Cl. CPC A61B 17/7216; A61B 2090/064

(57) ABSTRACT

Various implementations include implants and related methods for moving bone. Certain implementations include an implant for moving bone in a patient's body, the implant including: an implantable biocompatible housing; a first adjustment rod at least partially overlapping the implantable biocompatible housing; a driver configured to drive the first adjustment rod to enable movement of the first adjustment rod relative to the housing; and a load sensor positioned within the housing and configured to indicate a load applied by the driver on the first adjustment rod.

### TECHNICAL FIELD

This disclosure generally relates to biocompatible implants. More particularly, the disclosure relates to implants for moving bone in a patient's body.

### BACKGROUND

Implantable bone adjustment systems can beneficially treat a variety of conditions. For example, implantable bone adjustment systems can be used for purposes of distraction osteogenesis (also known as distraction callotasis and osteodistraction) in applications such as: post osteosarcoma bone cancer; cosmetic lengthening (both legs-femur and/or tibia) in short stature or dwarfism/achondroplasia; lengthening of one limb to match the other (congenital, post-trauma, post-skeletal disorder, prosthetic knee joint), nonunions, etc. Additionally, implantable bone adjustment systems can be used in treatment of various additional conditions and ailments such as scoliosis or osteoarthritis (e.g., knee osteoarthritis). Additional examples of treatment applications for implantable bone adjustment systems are described in U.S. patent application Ser. No. 16/298,339 (filed Mar. 11, 2019) and U.S. patent application Ser. No. 13/370,966 (filed Feb. 14, 2011), which are incorporated herein by reference in their entirety.

### SUMMARY

The needs above, as well as others, are addressed by embodiments of implants and related methods described in this disclosure. All examples and features mentioned below can be combined in any technically possible way.

Various implementations include implants for moving bone in a patient's body, related systems, and methods. Certain implementations include a biocompatible implant with an integrated load sensor.

In particular aspects, an implant for moving bone in a patient's body includes: an implantable biocompatible housing; a first adjustment rod at least partially overlapping the implantable biocompatible housing; a driver configured to drive the first adjustment rod to enable movement of the first adjustment rod relative to the housing; and a load sensor positioned within the housing and configured to indicate a load applied by the driver on the first adjustment rod.

In additional particular aspects, an implant for moving bone in a patient's body includes: an implantable biocompatible housing having a first cavity; a first adjustment rod at least partially contained within the first cavity; a driver configured to drive the first adjustment rod to enable movement relative to the housing, wherein the driver includes: a magnetic actuator configured to be actuated by a magnetic field external to the patient's body; and a driven gear system coupled with the magnetic actuator; and a load sensor positioned between the driven gear system of the driver and the first adjustment rod, the load sensor for indicating a load applied by the driver on the first adjustment rod.

In further particular aspects, a method includes: causing an implanted distraction device to expand using an external control device; measuring load sensor data with a load sensor of the implanted distraction device; and determining, using the load sensor data, an amount of movement of bone in the patient's body.

In additional particular aspects, a method of intermedullary adjustment of a patient's bone is performed using an implant described according to aspects of the disclosure.

Implementations may include one of the following features, or any combination thereof.

In particular aspects, the implant is configured to be wirelessly communicatively coupled to an external control device for transmitting data from the load sensor.

In certain cases, the implant includes a wireless transmitter and one or more processors configured to cause the wireless transmitter to transmit data from the load sensor.

In particular implementations, the implantable biocompatible housing comprises a first cavity defining an opening; wherein the first adjustment rod is at least partially disposed within the first cavity, wherein the load sensor is positioned between the driver and the opening.

In some aspects, the driver includes a magnetic actuator configured to be actuated by a magnetic field external to the patient's body.

In certain implementations, the implant has a proximal end and a distal end, wherein the driver further includes a driven gear system coupled with the magnetic actuator, wherein the load sensor is proximal to the driver, and wherein the driven gear system is distal to the load sensor. In some aspects, the interface between the housing and the driven gear system has a slip fit enabling translation of force measurement.

In particular cases, the load sensor includes a bi-directional load sensor coupled with the driver and configured to indicate both a compressive load and a tensile load applied by the driver to the first adjustment rod.

In some aspects, the load sensor is rotationally fixed relative to the housing. In certain examples, the rotationally fixed load sensor prevents rotation.

In certain implementations, the load sensor includes a button-type load cell. In certain examples, the button-type load cell is configured to rotate.

In particular aspects, the load sensor is configured to monitor the load applied by the driver on the first adjustment rod during adjustment of the first adjustment rod.

In some cases, in a portion of the housing in which the load cell is disposed, the housing has a non-circular cross-section that limits rotation of the load cell. In certain examples, the non-circular cross-section is oblong or hexagonal.

In certain aspects, the implant further includes a coupler holding the load cell in the portion of the housing. In certain examples, the coupler includes a snap ring or an o-ring.

In particular implementations, the load sensor includes a bi-directional load sensor coupled with the driver and configured to indicate both a compressive load and a tensile load applied by the driver to the first adjustment rod.

In some aspects, a proximal end of the first adjustment rod engages a lead screw and is configured to move with the lead screw.

In particular cases, the implant is an intermedullary implant.

In certain aspects, the implant is configured to aid in treatment of a limb length discrepancy or a bone defect in the patient's body.

In some cases, the load cell is configured to rotate with the driver relative to the housing.

In particular implementations, the load sensor includes a bi-directional load sensor coupled with the driver and configured to indicate both a compressive load and a tensile load applied by the driver to the first adjustment rod.

In some aspects, a proximal end of the first adjustment rod engages a lead screw and is configured to move with the lead screw.

In particular cases, the load sensor enables determination of the amount of movement of bone in the patient's body without imaging of the bone in the patient's body.

In certain implementations, the external control device is located proximate to the patient's body during expansion of the implanted distraction device.

In some cases, the external control device is remote relative to the patient's body during expansion of the implanted distraction device.

In particular aspects, determining the amount of movement of the bone in the patient's body includes converting the load sensor data into an estimated bone movement value.

In some implementations, the estimated bone movement value compensates for a margin of error.

In certain cases, the load sensor is connected with an external control device and is configured to wirelessly transmit load data to the external control device. In some examples, the external control device is located proximate to the patient or remote relative to the patient. In additional examples, the external control device includes an actuator for communicating with the driver in the implanted distraction device.

In certain implementations, the implants and methods enable enhanced lengthening rates for a patient.

Two or more features described in this disclosure, including those described in this summary section, may be combined to form implementations not specifically described herein.

The above presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

The details of one or more implementations are set forth in the accompanying drawings and the description below. Other features, objects and benefits will be apparent from the description and drawings, and from the claims.

### BRIEF DESCRIPTION OF THE DRAWINGS

FIG. 1 shows a perspective view of an intermedullary implant according to various implementations.

FIG. 2 shows a cross-section of an implant such as the implant in FIG. 1 according to various implementations.

FIG. 3 is a close-up cross-sectional view of a portion of the implant of FIGS. 1 and 2 according to various implementations.

FIG. 4 is a force/load diagram of the implant of FIGS. 1-3, according to various implementations.

FIG. 5 shows a cross-section of a portion of an implant according to various additional implementations.

FIG. 6 is a force/load diagram of the implant of FIG. 5 according to various implementations.

FIG. 7 shows a cross-section of a portion of an implant according to various implementations.

FIG. 8 shows a close-up cross-sectional view of a portion of the implant in FIG. 7 according to various implementations.

FIG. 9 shows a partially transparent perspective view of a portion of an implant according to various implementations.

FIG. 10 shows a cross-section of a portion of the implant in FIGS. 7-9, according to various additional implementations.

FIG. 11 shows a partially transparent perspective view of a portion of an implant according to various implementations.

FIG. 12 shows a cross-section of a portion of the implant in FIG. 11 according to certain implementations.

FIG. 13 is a force/load diagram of the implant of FIG. 12 according to various implementations.

FIG. 14 is a cross-section of an implant, including force/load indicators, according to various additional implementations.

FIG. 15 is a flow diagram illustrating processes in a method according to various implementations.

FIG. 16 shows the internal components of an external adjustment device for non-invasively adjusting a distraction and compression device according to various implementations.

FIGS. 17 and 18 show external adjustment devices in configurations for adjusting a distraction and compression device implanted within a femur, and within a tibia, respectively, in accordance with various implementations.

It is noted that the drawings of the various implementations are not necessarily to scale. The drawings are intended to depict only typical aspects of the disclosure, and therefore should not be considered as limiting the scope of the implementations. In the drawings, like numbering represents like elements between the drawings.

### DETAILED DESCRIPTION

Implantable bone adjustment systems have significantly enhanced the lives of patients, for example, enabling dynamic, long term bone adjustment without significant intrusion in the patient's daily life. However, certain implantable bone adjustment systems may benefit from feedback on forces applied by the implant, for example, in real time.

This disclosure provides, at least in part, implants for moving bone in a patient's body, and methods that beneficially incorporate such implants to move bone. These implants include a load sensor within the implant housing that is configured to directly or indirectly indicate a load applied by the implant to the patient's bone(s). In particular examples, the implant includes a housing having a driver that is configured to drive an adjustment rod, and a load sensor positioned within the housing that is configured to indicate a load applied by the driver on the adjustment rod.

The various disclosed implementations can improve patient outcomes when compared with conventional implantable adjusters. The disclosed implementations can provide adaptability in adjusting bone positioning, enhancing one or both of intraoperative and postoperative engagement with the device. Additionally, the disclosed implementations can provide feedback about adjustment protocols for a particular patient and/or a group of patients to enhance adjustment outcomes for the individual and/or group. The implants described according to various implementations can also reduce health risks for patients when compared with conventional approaches, for example, allowing more aggressive (shorter) adjustment protocols and mitigating the need for imaging as a means of adjustment feedback. Further, the force sensors can provide feedback to tune an amount of therapy provided to the patient (e.g., by directly measuring force applied in addition to or instead of measuring other indirect parameters).

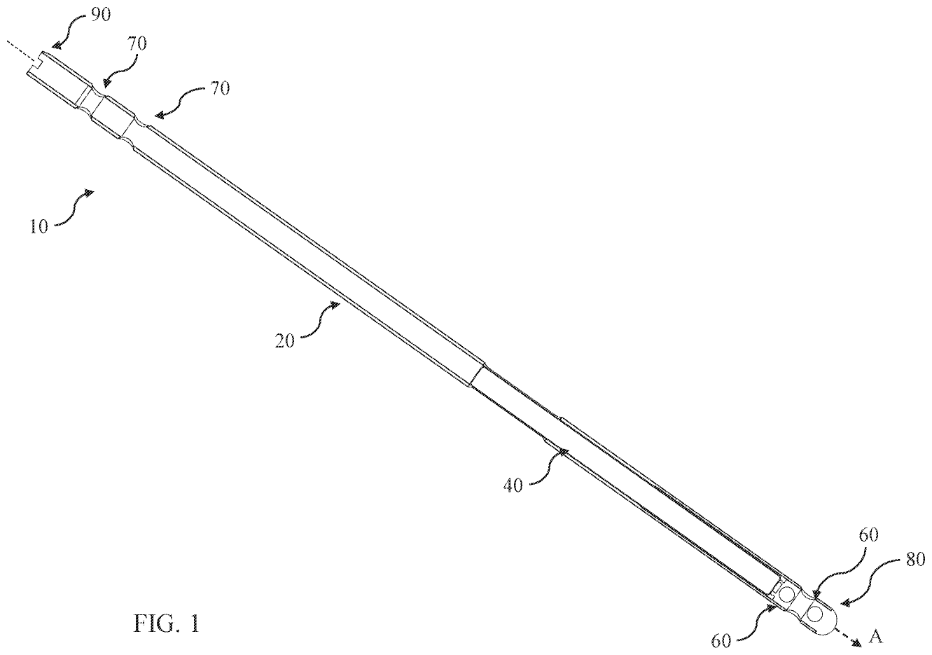

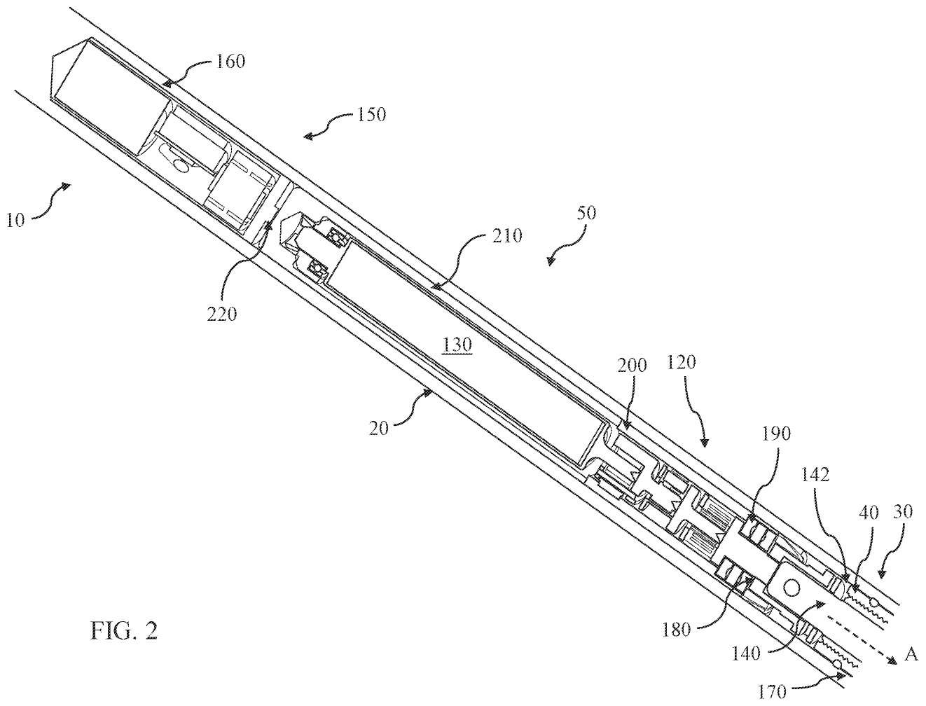

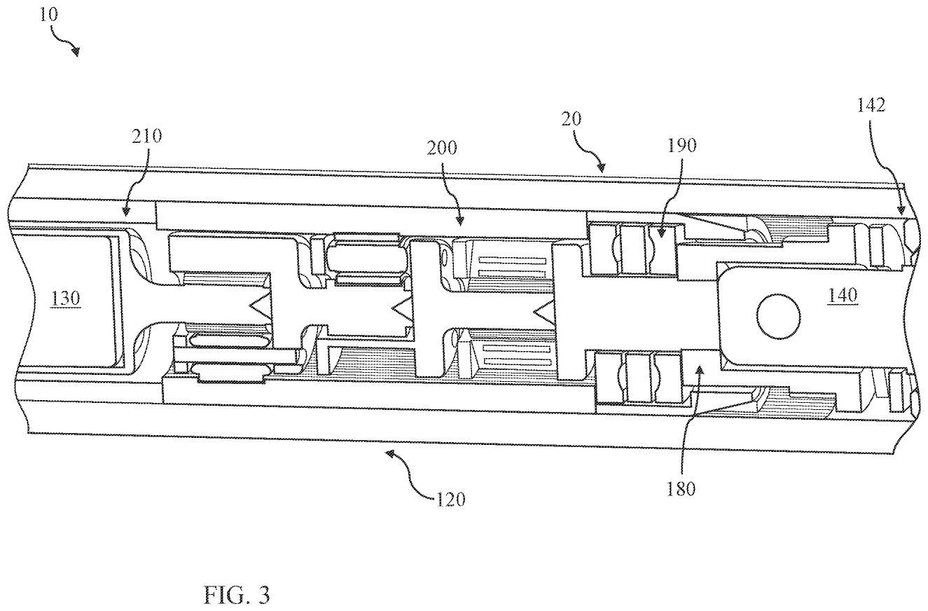

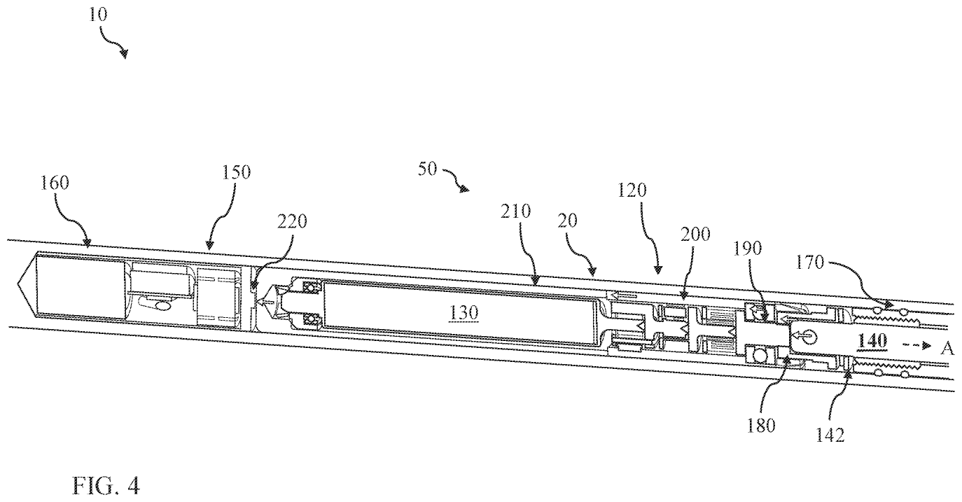

FIG. 1 shows a schematic perspective view of an implant 10 for moving bone in a patient's body according to various implementations. FIG. 2 shows a cross-sectional view of the implant 10. FIG. 3 shows a close-up view of a portion of the implant 10. FIG. 4 illustrates a schematic markup of load transfer locations (interfaces) in the implant 10. In particular implementations, the implant 10 (and other implants depicted and described herein) is configured for intermedullary placement in a patient, e.g., to aid in treatment of one or more patient conditions. In certain examples, the implant 10 can be used in a method of intermedullary adjustment of a patient's bone. In a particular example, the implant 10 (and other implant(s) herein) is configured to aid in treatment of a limb length discrepancy and/or a bone defect in the patient's body. In other implementations, the techniques described herein can be applied in non-intermedullary uses, such as for use with extramedullary plates, in the correction of scoliosis or for other uses.

With reference to FIGS. 1-4, in various implementations, the implant 10 includes an implantable biocompatible housing (or, “housing”) 20. The housing 20 can include a first cavity 30, with a first adjustment rod 40 at least partially contained in the first cavity 30. A drive mechanism (or, driver) 50 is configured to drive the first adjustment rod 40 to enable movement, e.g., translation (also called “distraction”) of the rod 40 axially relative to the housing 20 (relative to primary axis A). While described in terms of translation (distraction) in certain implementations, it is understood that additional implementations of an implant can include a driver configured to translate and/or rotate the first adjustment rod 40 and/or additional adjustment rods such as described in U.S. Patent Application No. 63/342,921 (Implant with Multi-Modal Adjustment, filed May 17, 2022), which is entirely incorporated by reference herein.

In various implementations, the housing 20 and the first adjustment rod 40 are each configured for coupling to a patient. In various implementations, the first adjustment rod 40 includes one or more holes 60 for passing an anchor with which to secure the implant 10 to the patient (e.g., to bone), as shown in FIG. 1. The housing 20 can also include one or more holes 70 for passing an anchor with which to secure the implant to the patient (e.g., to bone). Spacing of holes 60 relative to a distal end 80 of the implant 10, spacing of holes 70 relative to a proximal end 90 of the implant 10, dimensions of the holes 60, 70, and other dimensional aspects of the holes 60, 70 can be tailored for particular patient parameters and/or treatment profiles. In certain aspects, the anchors described herein can include bone screws or other bone fixators or connectors. Examples of bone screws and/or dimensional aspects of the holes 60, 70 are described in U.S. patent application Ser. No. 16/298,339, entirely incorporated by reference herein.

In certain cases, the first adjustment rod 40 can include two distinct portions, which can be telescopically actuated to apply translational pressure on the patient's bone. For example, portions of the first adjustment rod 40 can be configured to translate telescopically, e.g., as described with reference to FIGS. 1-5 of U.S. patent application Ser. No. 16/298,339, previously incorporated by reference herein. In certain cases, the drive mechanism includes at least one driver 50 that is configured to be actuated by an external control device. In some cases, the external control device includes an external actuator for communicating with the driver 50 from a location external to the patient's body, e.g., a magnetic controller and/or other wireless controller. In particular cases, the driver 50 is configured to control translation of the first adjustment rod 40. In certain implementations, the driver 50 includes a gear system (e.g., a driven gear system having with planetary gears) 120 for driving movement of the first adjustment rod 40. In some examples, the driven gear system 120 is coupled with a magnetic actuator (e.g., permanent magnet) 130 that is rotationally coupled to the driven gear system 120. However, other driving elements are suitable in place of a magnet. For example, in addition to or instead of the magnet-based driving, one or more of the driving elements can take the form of an implanted electric motor. The implanted electric motor can be powered by an external power source (e.g., via a radiofrequency link, via an inductive connection, or via another technique). The implanted electric motor can be powered by an implanted power source (e.g., a battery, which may be charged by the external power source). The implanted power source may be within the implant (e.g., within a housing thereof) or separate from the implant and coupled to the implant via a cable. In certain cases, the driven gear system 120 is mounted with radial bearings and/or thrust bearings to a lead screw 140 that drives translation of the first adjustment rod 40. That is, a proximal end 142 of the first adjustment rod 40 engages the lead screw 140 and is configured to move with the lead screw 140. As noted herein, driver 50 can be configured to control translation of the first adjustment rod 40. In some cases, the driven gear system 120 can have a partially (or fully) slip-fit connection with the housing 20 that enables force to be transferred to a load cell 150, e.g., to provide accurate measurement of the load applied by the driven gear system 120 to the lead screw 140.

In particular cases, the implant 10 further includes a load sensor 150 positioned within the housing 20 and configured to indicate a load applied by the driver 50 to the first adjustment rod 40. In some cases, the load sensor 150 is configured to monitor the load applied by the driver 50 on the first adjustment rod 40 during adjustment of the first adjustment rod 40. As descried according to various implementations, the load sensor 150 can include a bi-directional load sensor that is coupled with the driver 50 and is configured to indicate both a compressive load and a tensile load applied by the driver 50 to the first adjustment rod 40. In additional implementations, the load sensor 150 can include a single-directional load sensor coupled with the driver 50 that is configured to indicate one of a compressive load or a tensile load applied by the driver 50 to the first adjustment rod 40. In certain implementations, the load sensor 150 is rotationally fixed relative to the housing 20, e.g., to prevent rotation during driving of the first adjustment rod 40. In additional implementations, the load sensor 150 is able to rotate relative to the housing 20. In a particular example illustrated in FIG. 2, the load sensor 150 includes a button-type load cell, which in certain cases, is able to rotate relative to the housing 20.

In particular implementations, for example as illustrated in FIGS. 2 and 4, the implant 10 further includes a communication device 160 that is configured to transmit data from the load sensor 150. In certain cases, the communication device 160 includes a wireless transmitter including and/or coupled with a processor or set of processors that is configured to cause the transmitter to transmit data from the load sensor 150. In various implementations, the communication device 160 enables wireless communication with an external control device, e.g., by enabling transmission of data such as load data from the load sensor 150. In particular examples, the load sensor 150 is connected with an external control device and is configured to wirelessly transmit load data to the external control device. In some examples, the external control device is located proximate to the patient or remote relative to the patient. In additional examples, the external control device includes an actuator for communicating with the driver in the implant 10.

Turning to FIGS. 2-4, in particular implementations, the housing 20 is shown with the cavity 30 that has an opening 170. In certain cases, the first adjustment rod 40 is at least partially disposed within the cavity 30. In additional implementations, the load sensor 150 is positioned within the housing 20, where the driver 50 is located between the load sensor 150 and the opening 170. That is, in various implementations, the load sensor 150 is proximal to the driver 50 (i.e., closer to the proximal end 90 of the implant 10), and the driver 50 is distal to the load sensor 150 (i.e., closer to the distal end 80 of the implant 10).

In particular implementations, as illustrated in the example depiction in FIGS. 2-4, the implant can include a button-type load sensor 150 (or, load cell) that is configured to detect a load applied to the first adjustment rod 40. In such cases, the load sensor 150 can be configured to rotate relative to the housing 20, e.g., about the primary axis (A) of the implant 10. In particular, FIG. 4 illustrates an example of force/load transfer between components in the implant 10 as detected by the load sensor 150. The force (load) transfer diagram illustrates reactive forces that are transferred to the load sensor 150 caused by actuation of the driver 50. As shown, load is transferred at a first stage (1) from the lead screw 140 to a coupler 180, at a second stage (2) from the coupler 180 to a thrust bearing 190, at a third stage (3) from the thrust bearing 190 to a ring gear 200, at a fourth stage (4) from the ring gear 200 to a magnet housing 210 for the magnetic actuator (magnet) 130, and at a fifth stage (5) from the magnet housing 210 to the load sensor 150 (e.g., to a button interface 220 at the load sensor 150). In particular implementations, the button-type load sensor 150 and configuration illustrated in FIGS. 2-4 is suited for detecting a compressive load. A button-type load sensor 150 can have a cylindrical shape. In this example, the load sensor 150 is configured to detect the load applied by the driver 50 on the first adjustment rod 40, and in particular cases, transmit that load data to an external control device (e.g., via communication device 160 and associated processor(s)). In certain cases, the processor(s) can be coupled with memory for storing load data locally. In additional cases, the external control device can include memory for storing load data.

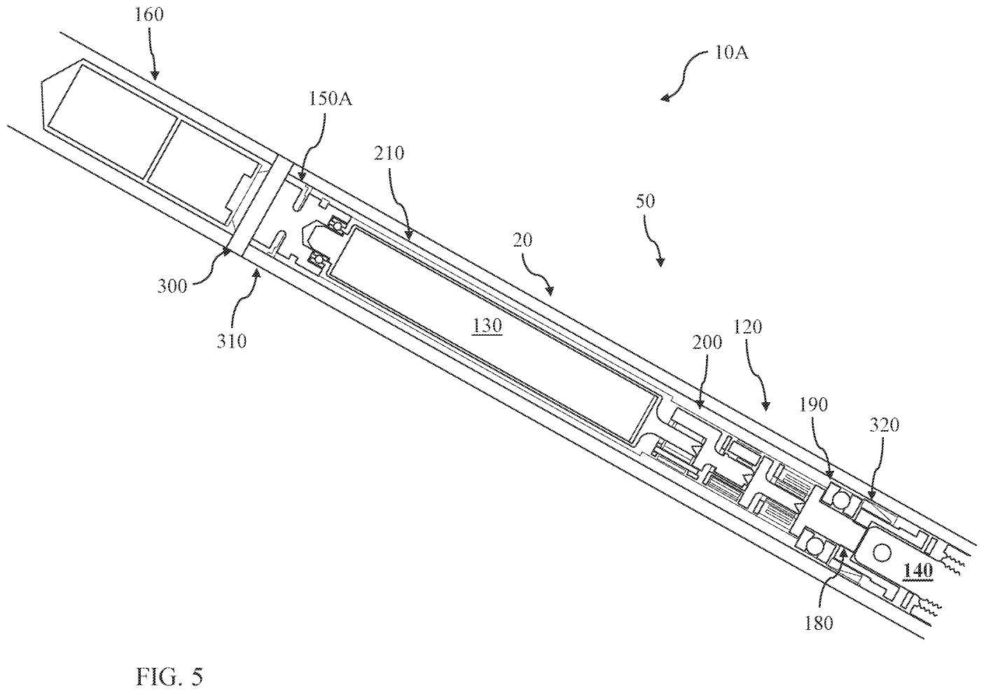

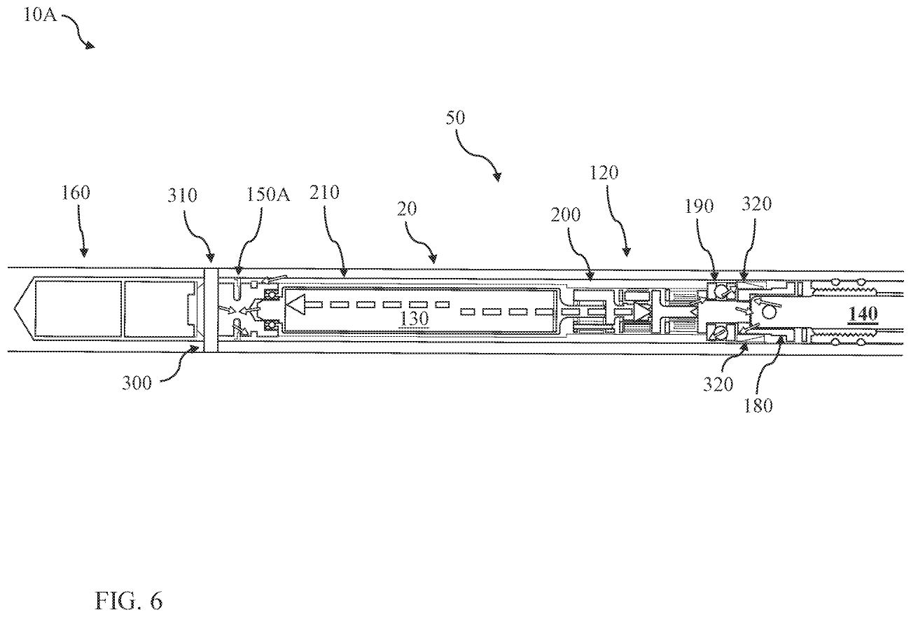

FIGS. 5 and 6 illustrate another implementation of an implant 10A according to the disclosure. FIG. 5 shows a cross-section of the implant 10A, and FIG. 6 is a schematic force/load transfer diagram of the implant 10A. According to certain implementations, a load sensor 150A in the implant 10A is rotationally fixed relative to housing 20. That is, in certain implementations, a coupler 300 holds the load sensor 150A in a portion 310 of the housing 20. In certain cases, the load sensor 150A anchors to the housing 20 with a coupler 300 that includes one or more pins, screws, bolts, etc. According to some implementations, the load sensor 150A directly contacts the magnet housing 210 for the magnetic actuator 130. In particular cases, the fixed position of the load sensor 150A (e.g., via coupler 300 such as pin(s), screw(s), bolt(s), etc.) enables the load sensor 150A to detect both compressive and tensile loads translated from the adjustment rod 40. In certain cases, the coupler 300 can include a set of spring-loaded pins (e.g., two pins) that mate with recesses in the housing 20, thereby retaining the load sensor 150A in the housing 20 without extending through the outer surface of the housing 20. In any case, as noted herein, the implant 10A can be configured to detect one or both of compressive and tensile loads, e.g., to aid in determining operating parameters of the implant 10A in the patient's body. FIG. 6 illustrates load transfer in both compressive and tensile modes. Load transfer in compressive mode is similar to the description in FIG. 4. In tensile mode, tension on the lead screw 140 is transferred to coupler 180, which is then transferred to the opposite side of the thrust bearing 190, then to the edge of a wedge lock 320, from the wedge lock 320 to the ring gear 200/magnet housing 210 interface, and then from the magnet housing 210 to the load sensor 150A.

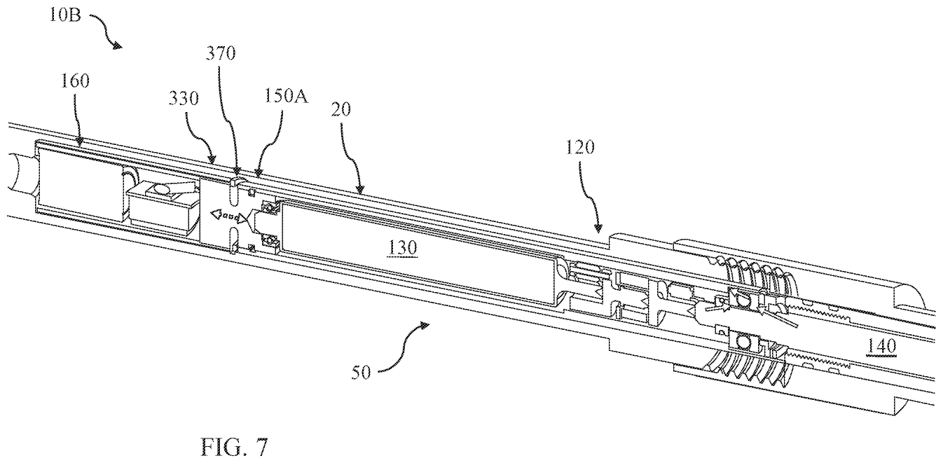

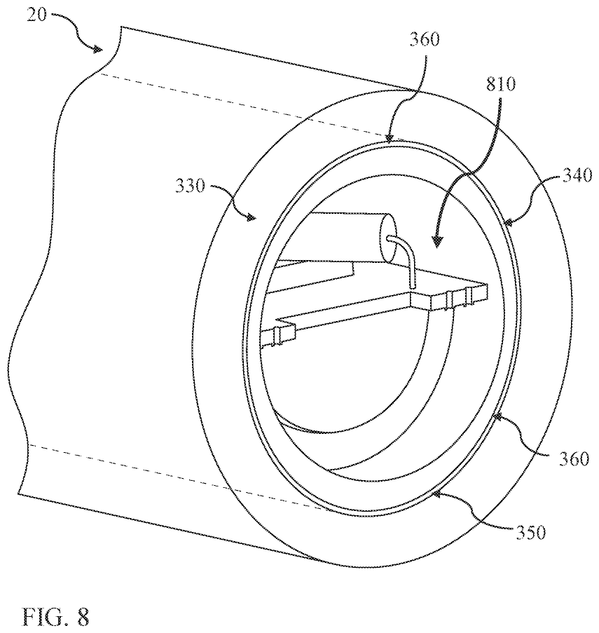

FIGS. 7-10 illustrate another implant 10B according to additional implementations. As described herein, a portion of the housing 20 in the implant 10B is configured with a torque limiting (or rotation limiting) feature. In one example, as shown in FIG. 7, a portion 330 of the housing 20 in implant 10B houses a load sensor 150A, such as a bi-directional load sensor similar to load sensor 150A in FIG. 6. In these embodiments, the portion 330 of the housing 20 in which the load sensor 150A is disposed has a non-circular cross-section 340 that limits rotation of the load sensor 150A. In certain cases, the load sensor 150 is approximately non-circular, and in particular cases, complements the non-circular cross-section 340 of the housing. In other cases, the load sensor 150 is approximately circular, and interferes with the non-circular cross-section 340 of the portion 330 of housing 20. FIG. 8 shows a close-up perspective of the non-circular cross-section 340 illustrating example rotation limiting features of the housing 20. That is, an inner wall (surface) 350 of the housing 20 is non-circular (e.g., oblong, hexagonal, etc.) and an outer wall (surface) 360 of the load sensor 150A is non-circular, or approximately circular, causing interference between the two surfaces 350, 360. In certain implementations, a coupler 370 holds the load sensor 150A in the portion 330 of the housing 20. The coupler 370 can include a snap-ring, o-ring, or other retaining feature that is at least partially circumferentially disposed about a recess (or, notch) 380 in the outer surface 360 of the load sensor 150A.

In addition, FIG. 8 illustrates circuitry 810. The circuitry 810 can be electrically connected to one or more of the load sensors 150. The circuitry 810 can include one or more power storage components, such as one or more batteries or capacitors. The power storage components can provide power for one or more other components of the circuitry 810 or other components of the implant 10, such as one or more of the load sensors 150. The circuitry 810 can include one more power generation components, such as may be used to obtain power from movement of the implant 10 (e.g., as caused by movement of the recipient of the implant) or conditions around the implant (e.g., temperature). The circuitry 810 can include one or more microcontrollers (e.g., one or more processors, such as central processing units) and/or memory circuits. The circuitry 810 can also include one or more components configured for bi-directional or uni-directional. communication with an external device. The communication can include receiving power from an external device (e.g., to charge one or more of the power storage components). The communication can include receiving commands from an external device. The communication can include providing data to an external device (e.g., load data from the one or more load sensors). The circuitry 810 can include one or more aspects, configurations, or components as described in US 2020/0253588 (filed 2020 Feb. 7), U.S. Pat. No. 11,389,111 (filed 2019 Jan. 17), and US 2019/0254712 (filed 2019 Apr. 30), which are each hereby incorporated herein by reference in their entirety for any and all purposes.

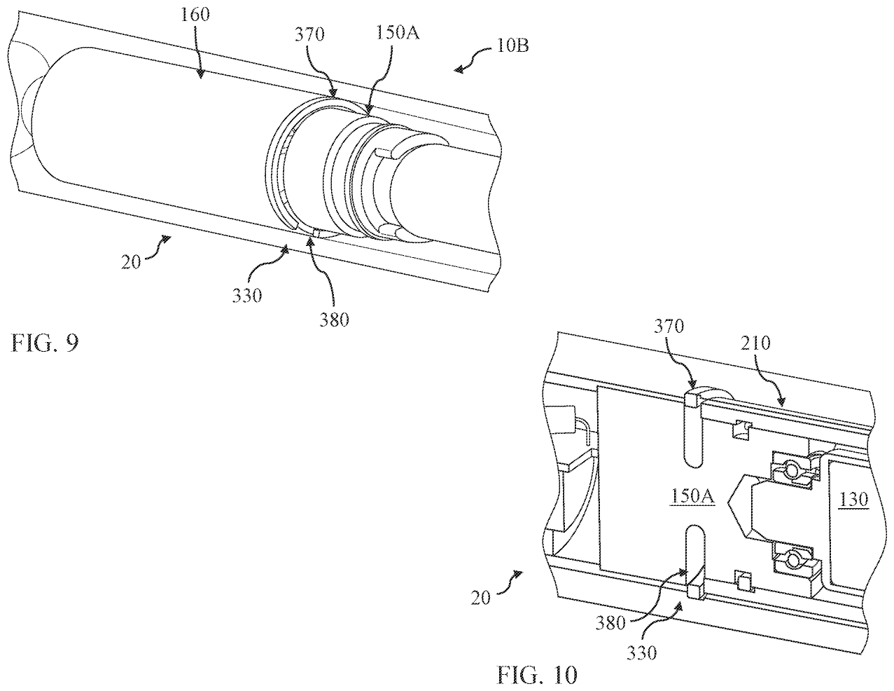

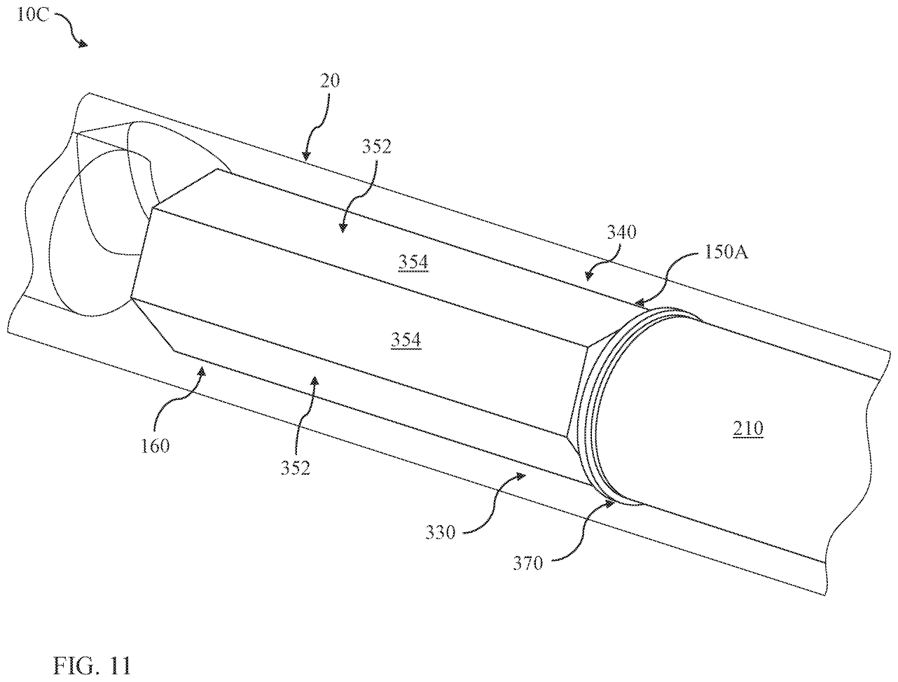

FIGS. 9 and 10 illustrate partially transparent and cross-sectional views, respectively, of a portion of the implant 10B that includes the coupler 370. In certain implementations, the coupler 370 aids in retaining the load sensor 150A in the housing 20, e.g., axially, or in the proximal-distal direction.

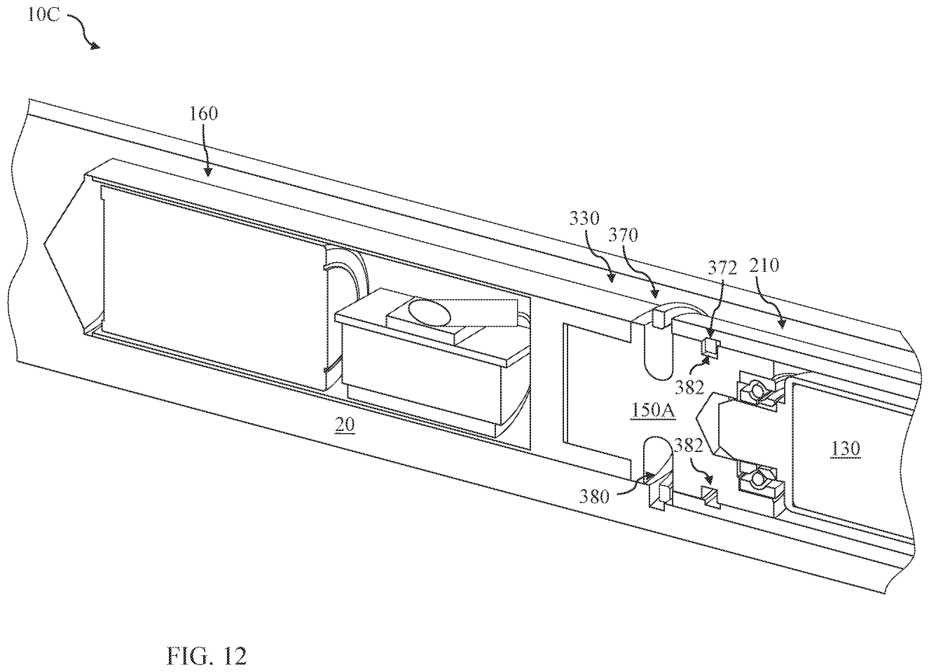

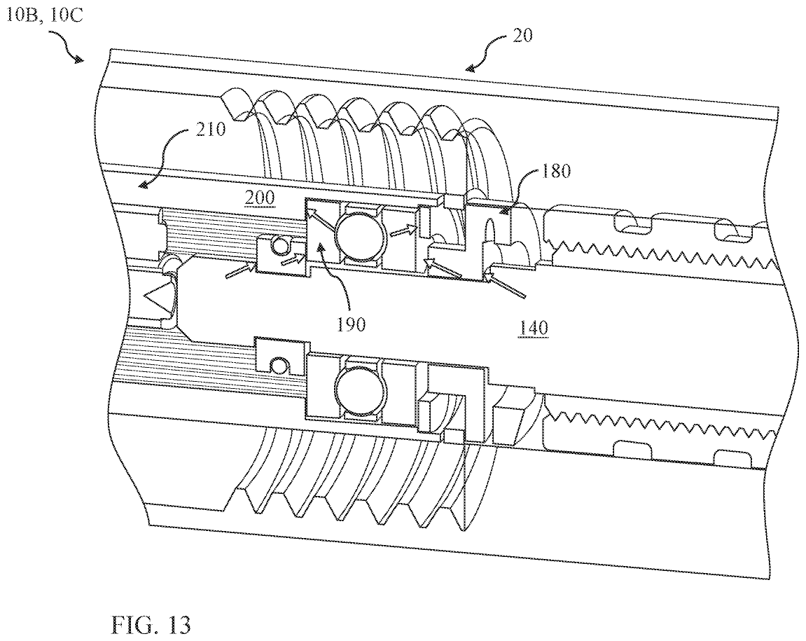

FIGS. 11 and 12 show partially transparent perspective, and a cross-sectional views, respectively, of another implant 10C according to various implementations. Similar to implant 10B, in implant 10C, a portion 330 of the housing 20 houses a load sensor 150A, such as a bi-directional load sensor. In these embodiments, the portion 330 of the housing 20 in which the load sensor 150A is disposed has a non-circular cross-section 340 that limits rotation of the load sensor 150A. In a particular example, the inner wall (surface) 350 of the housing 20 includes at least one flat section 352, and in more particular cases, includes a plurality of flat sections 352. In particular cases, flat sections 352 of the inner wall (surface) 350 complement flat sections 354 of the outer surface 360 of the load sensor 150A. In certain examples, the inner wall (surface) 350 of the housing has an approximately hexagonal cross-section. In additional implementations, the outer surface 360 of the load sensor 150A is circular or approximately circular, causing interference between the two surfaces 350, 360. In certain implementations, a coupler 370 holds the load sensor 150A in the portion 330 of the housing 20. The coupler 370 can include a snap-ring, o-ring, or other retaining feature that is at least partially circumferentially disposed about the recess (or, notch) 380 in the outer surface 360 of the load sensor 150A. In certain implementations, the coupler 370 aids in retaining the load sensor 150A in the housing 20, e.g., axially, or in the proximal-distal direction. In additional implementations, a second coupler 372 (e.g., a snap ring or o-ring) is positioned between a second recess (or, notch) 382 in the load sensor 150A and the magnet housing 210, e.g., to secure the load sensor 150A to the magnet housing 210 and connected ring gear 200.

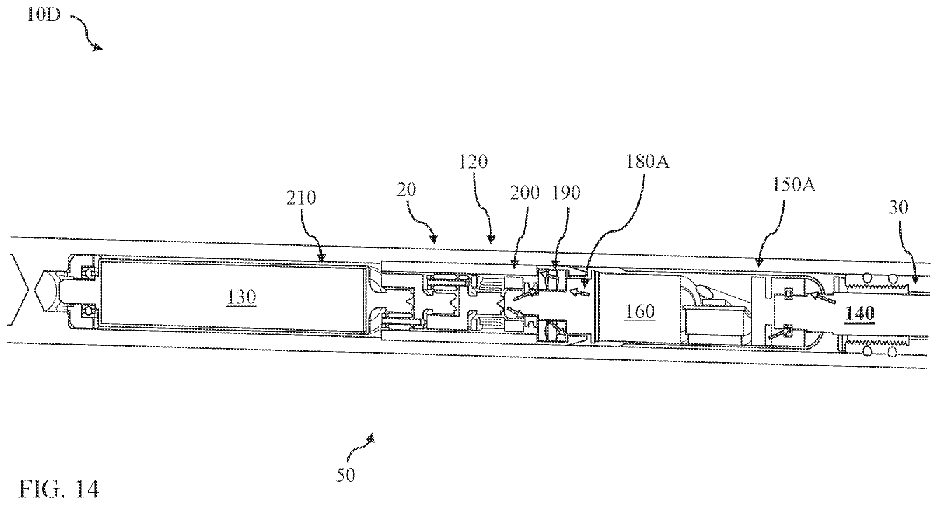

FIG. 13 is an example force loading diagram for implant 10B and/or implant 10C according to various implementations. As illustrated, the bi-directional load sensor 150A is positioned to detect force applied to the interface of the magnet housing 210 and ring gear 200 from either direction (e.g., pulling or pushing). Regardless of the rotation-limiting shape of the load sensor and the housing, the ability to detect force loading will be approximately the same between both implants 10B and 10C.

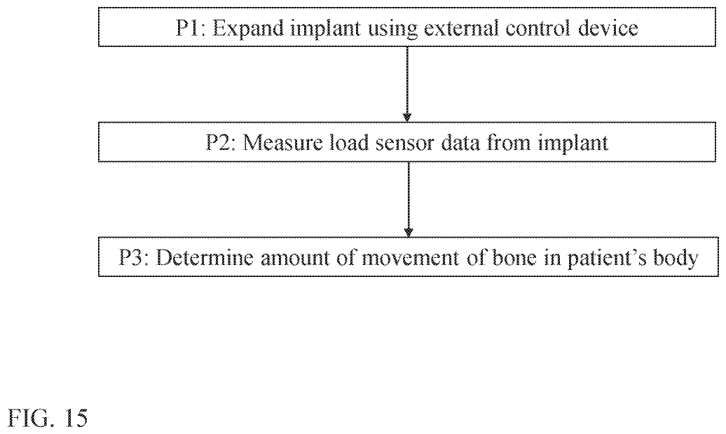

FIG. 14 illustrates a cross-section of an additional implant 10D according to various additional implementations. In contrast to certain other implants shown and described herein, implant 10D includes a load sensor 150A (e.g., bi-directional load sensor) that is positioned between (e.g., axially between, in the proximal-distal direction) the driven gear system 120 of the driver 50 and the first adjustment rod 40 for example, to indicate a load applied by the driver 50 on the first adjustment rod 40. In particular implementation, the load sensor 150A is directly coupled with the driver 50 (e.g., adjacent driven gear system 120) and is configured to rotate with the driver 50 relative to the housing 20. As with other implementations utilizing load sensor 150A, the implant 10D is configured to detect both compressive and tensile loads on the first adjustment rod 40 as indicated by tensile load arrows (left-to-right) and compressive load arrows (right-to-left) in FIG. 14. In these implementations, compressive loads can be detected via coupler 180A that is directly connected to the back (opposing) side of the load sensor 150A from the connection with the lead screw 140.

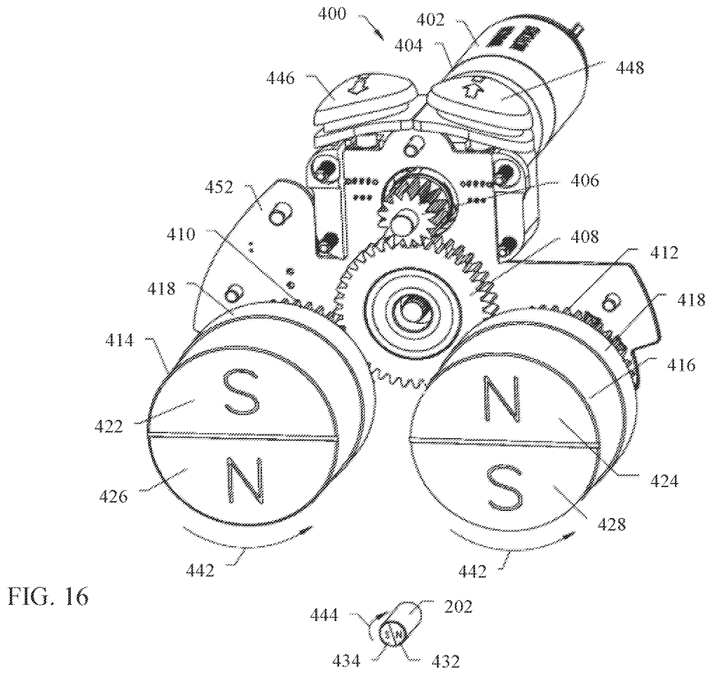

FIG. 15 is flow diagram illustrating a method of using an implant (e.g., implant 10 or any of implants 10A-D) according to certain implementations. In various implementations, the process is performed while the implant is implanted within a patient, e.g., coupled to one or more bone segments in the patient. In a first process, P1: the implant 10 is expanded using an external control device such as a controller described herein. In certain cases, the external control device is located proximate to the patient's body during expansion of the implant 10. In other cases, the external control device is remote relative to the patient's body during expansion of the implant 10. Either contemporaneously with process P1 or following expansion in P1, in process P2, load sensor data is measured with a load sensor 150 of the implant 10 (e.g., load sensor 150 and/or load sensor 150A). Following measuring of the load sensor data with load sensor 150, in process P3, an amount of movement of bone in the patient's body is determined. In certain cases, determining the amount of movement of the bone in the patient's body includes converting the load sensor data into an estimated bone movement value. In some examples, a data model is used to estimate the bone movement value, e.g., applying a correspondence table or other calculation to account for load sensor data, patient-specific data, implant-specific data, etc. In particular cases, the estimated bone movement value compensates for a margin of error. In some examples, the movement is determined based on a known or estimated amount of load over time needed to move a particular amount of bone. The amount can be adjusted based on known movement of the patient. For example, an initial image of the region of interest can be taken, the implant can be actuated while load is measured, and a subsequent image can be taken. The amount of load can be correlated with changes in bone visible by a comparison between the initial and subsequent images. In some examples, changes in an amount of load can be used to determine an amount of bone movement or determine when a next bone adjustment can be made.

In any case, the use of the load sensor (e.g., load sensor 150 and/or load sensor 150A) enables determination of the amount of movement of bone in a patient's body without imaging of that bone. That is, the implants shown and described herein can reduce reliance on imaging as feedback for bone adjustment in patients, potentially reducing the patient's exposure to imaging-based radiation and additional complications.

Any implant shown and described herein (e.g., implants 10, 10A, 10B, 10C, 10D) can be configured for intermedullary placement in a patient, e.g., to aid in treatment of a limb length discrepancy or a bone defect in the patient's body. In certain cases, the implants described and depicted herein can be used in a method of intermedullary adjustment of a patient's bone, e.g., by inserting the implant(s) into the patient's body and by actuating the implant(s) using a controller such as an external control device. As noted herein, further processes in the methods can include measuring load sensor data from a load sensor at the implant, and determining an amount of movement of bone in a patient's body using the load sensor data.

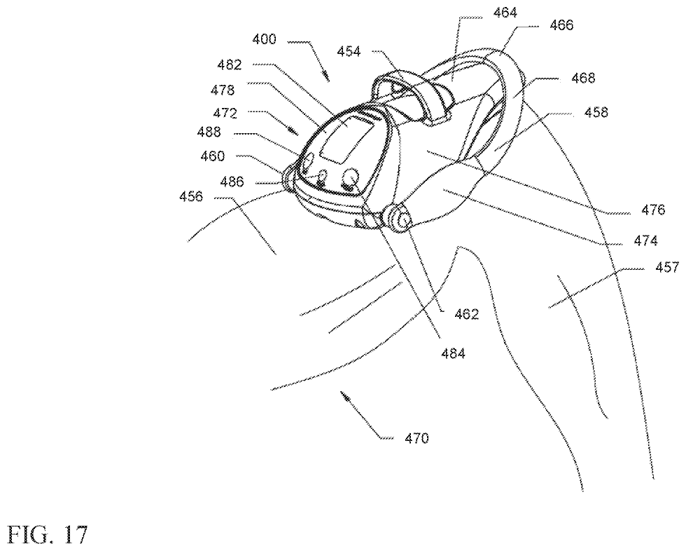

FIGS. 16-18 illustrate an external adjustment device 400 configured for applying a moving magnetic field to allow for non-invasive adjustment of the device 100 by turning a permanent magnet 202 within the device 100, as described. FIG. 16 illustrates the internal components of the external adjustment device 400, and for clear reference, shows the permanent magnet 202 of the device 100 without the rest of the assembly. The internal working components of the external adjustment device 400 may, in certain embodiments, be similar to those described in U.S. Patent Application Publication No. 2012/0004494, which is incorporated by reference herein. A motor 402 with a gear box 404 outputs to a motor gear 406. The motor gear 406 engages and turns a central (idler) gear 408, which has the appropriate number of teeth to turn a first magnet gear 410 and a second magnet gears 412 at identical rotational speeds. First and second magnets 414, 416 turn in unison with the first and second magnet gears 410, 412, respectively. Each magnet 414, 416 is held within a respective magnet cup 418 (shown partially). An exemplary rotational speed may be 60 RPM or less. This speed range may be configured to limit the amount of current density induced in the body tissue and fluids, to meet international guidelines or standards. As seen in FIG. 16, the south pole 422 of the first magnet 414 is oriented the same as the north pole 424 of the second magnet 416, and likewise, the first magnet 414 has its north pole 426 oriented the same as the south pole 428 of the second magnet 416. As these two magnets 414, 416 turn synchronously together, they apply a complementary and additive moving magnetic field to the radially-poled, permanent magnet 202, having a north pole 432 and a south pole 434. Magnets having multiple north poles (for example, two) and multiple south poles (for example, two) are also contemplated in each of the devices. As the two magnets 414, 416 turn in a first rotational direction 442 (e.g., counter-clockwise), the magnetic coupling causes the permanent magnet 202 to turn in a second, opposite rotational direction 444 (e.g., clockwise). The rotational direction of the motor 402 and corresponding rotational direction of the magnets 414, 416 is controlled by buttons 446, 448. One or more circuit boards 452 contain control circuitry for both sensing rotation of the magnets 414, 416 and controlling rotation of the magnets 414, 416.

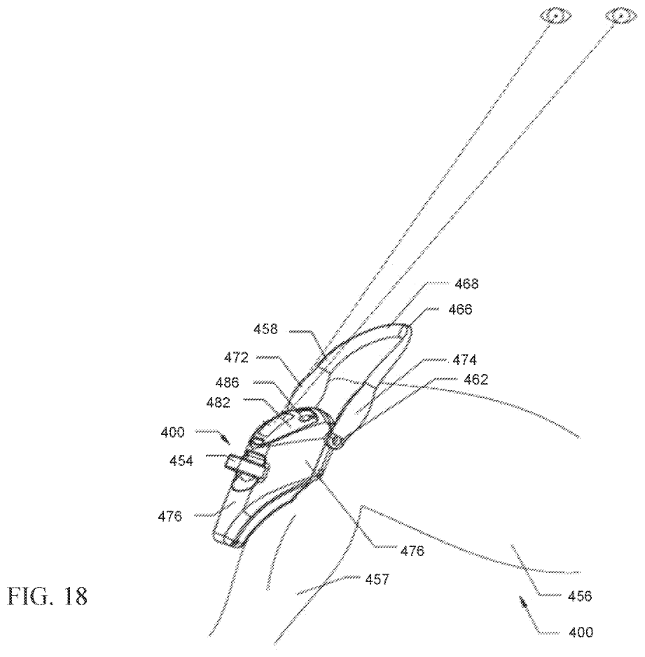

FIGS. 17 and 18 show the external adjustment device 400 for use with a device 100 placed in the femur (FIG. 17) or the tibia (FIG. 18). The external adjustment device 400 has a first handle 454 for carrying or for steadying the external adjustment device 400, for example, steadying it against an upper leg 456 (as in FIG. 17) or a lower leg 457 (as in FIG. 18). An adjustable handle 458 is rotationally attached to the external adjustment device 400 at pivot points 460, 462. Pivot points 460, 462 have lockable/unlockable mechanisms, such as a spring-loaded brake, ratchet or tightening screw, so that a desired angulation of the adjustable handle 458 in relation to housing 464 can be adjusted and locked in orientation. Adjustable handle 458 is shown in two different positions in FIGS. 17 and 18. In FIG. 17, adjustable handle 458 is set so that an apex 466 of a loop 468 rests against housing 464. In this position, patient 470 is able to hold onto one or both of grips 472, 474 while the adjustment procedure (for example transporting bone between 0.10 mm to 1.50 mm) is taking place. It is contemplated that the procedure could also be a lengthening procedure for a bone lengthening device or a lengthening procedure for a lengthening plate which is attached external to the bone. Turning to FIG. 18, when the bone transport device 100 is implanted in a tibia, the adjustable handle 458 may be changed to a position in which the patient 470 can grip onto the apex 466 so that the magnet area 476 of the external adjustment device 400 is held over the portion of the device 100 containing the permanent magnet 202. In both cases, the patient 470 is able to clearly view control panel 478 including a display 482. In a different configuration from the two directional buttons 446, 448, in FIG. 16, the control panel 478 includes a start button 484, a stop button 486 and a mode button 488. Control circuitry contained on circuit boards 452 may be used by the surgeon to store information related to specific aspects of each particular patient. For example, in some patients an implant may be placed antegrade into the tibia. In other patients the implant may be placed either antegrade or retrograde about the femur. In each of these three cases, it may be desired to move the bone either from distal to proximal or from proximal to distal. By having the ability to store information of this sort that is specific to each particular patient within the external adjustment device 400, the external adjustment device 400 can be configured to direct the magnets 414, 416 to turn in the correct direction automatically, while the patient need only place the external adjustment device 400 at the desired position and push the start button 484. The information of the maximum allowable bone transport length per day and maximum allowable bone transport length per session can also be input and stored by the surgeon for safety purposes. These may also be added via a memory storage unit (e.g., an SD card, USB device, etc.), or by wireless input (e.g., Bluetooth, WiFi, etc.). An additional feature is a camera at the portion of the external adjustment device 400 that is placed over the skin. For example, the camera may be located between first magnet 414 and second magnet 416. The skin directly over the implanted permanent magnet 202 may be marked with indelible ink. A live image from the camera is then displayed on the display 482 of the control panel 478, allowing the user to place the first and second magnets 414, 416 directly over the area marked on the skin. Crosshairs can be overlaid on the display 482 over the live image, allowing the user to align the mark on the skin between the crosshairs, and thus optimally place the external adjustment device 400.

Other external adjustment devices can be used to cause actuation of the distraction devices described herein. Such external adjustment devices include, for example, those described in U.S. Pat. No. 8,382,756 filed on Nov. 20, 2009, U.S. Pat. No. 9,248,043 filed Jun. 29, 2011, U.S. Pat. No. 9,078,711 filed on Jun. 6, 2012, U.S. Pat. No. 9,044,281 filed on Oct. 18, 2012, U.S. application Ser. No. 14/698,665 filed on Apr. 28, 2015, U.S. application Ser. No. 14/932,904 filed on Nov. 4, 2015, U.S. Ser. No. 16/004,099 filed on Dec. 12, 2016, and App. No. PCT/US2020/017338 filed on Feb. 7, 2020, all of which are incorporated herein by reference as if set forth in their entirety.

Further, any implant described herein can be part of an implantable adjustment system that incorporates an external remote controller (ERC) or other external control device. In certain cases, the ERC can include a magnetic handpiece, a controller (or control box, e.g., with a processor), and a power supply. In additional implementations, the ERC or other external control device can include an interface such as a user interface for enabling a medical professional to interact with the system including implant(s) described herein. Additional details of an ERC and interaction with implants are described in U.S. patent application Ser. No. 16/298,339, previously incorporated by reference herein.

Even further, the implants, associated systems and controllers can include a communication system for connecting devices (e.g., via wireless or hard-wired means), or integral with particular devices (e.g., ERC). The communication system can include a number of hard-wired and/or wireless communication systems, with certain wireless systems configured to communicate over Bluetooth, Bluetooth Low Energy (BLE), radio frequency (RF), Wi-Fi, and/or ultrasound. In additional implementations, the communication system can include an independent subscriber identity module (SIM) assigned to each implant. In further cases, the communication system is configured to communicate wirelessly with a remote control system and/or data gathering/analysis platform, e.g., via a cloud-based communication protocol.

In particular cases, each implant is individually programmable to control an amount of the adjustment of the patient's bone. For example, implants described herein may each include an individually programmable or adjustable component (e.g., programmable controller and/or gear ratio, thread pitch and/or count, etc.) to control the amount of adjustment of the patient's bone. In certain cases, distinct implants in a system can be programmed or otherwise designated to perform distinct adjustments.

In additional cases, the controller(s) described herein includes a smart device (e.g., smart phone, smart watch, tablet, etc.) configured to operate a control platform for adjusting the implants. In these instances, the control platform can include a software application (or “app”) configured to execute or otherwise run at a controller (e.g., ERC) for enabling control of one or more implants. According to certain implementations, the control platform enables control functions for one or more implants from a remote physical location relative to device 100. For example, the control platform can enable connection (e.g., network-based and/or cloud-based connection) between a system including the implant(s) described herein and a remote user such as a medical professional.

In all implementations described herein, the implant(s) can further include a feedback system in communication with one or more control devices (e.g., ERC and/or software application running control program). In certain cases, the feedback system provides feedback on a force response to the adjustment of the length of a given adjustment rod and/or rotation of a given adjustment rod. In certain cases, the feedback system includes a sensor onboard the implant, e.g., a sensor that is integrated with or coupled with the housing. Non-limiting examples of sensors can include a load cell, a piezo (piezoelectric) sensor, or an imaging sensor (e.g., optical sensor such as a camera, or an ultrasound sensor). Additional sensors that can be integrated in, or otherwise form part of the feedback system can include position and/or speed sensors (e.g., gyroscope/magnetometer, or inertial measurement unit (IMU)), temperature sensors and/or humidity sensors. In certain cases, the feedback system provides instructions to the controller (e.g., ERC) to modify actuation of a given implant based on the feedback on the force response.

In still further implementations, the sensor(s) in the feedback system described herein can be configured to provide data about a load exerted on an adjustment element, and/or a load exerted by the adjustment element on the patient's bone. The sensor(s) can also provide data about a tensile load between the implants and bone. In certain implementations, both tension and compression data are recorded by sensor(s) and provided to the feedback system for analysis and/or action (e.g., to adjust adjustment instructions). It is understood that tension and/or compression data detected by sensors, can represent an inferred or correlated indicator of the tension and/or compression applied to a device or component not physically in contact with the sensor. For example, the sensor on an instrument can be configured to detect tension at the instrument, while that torque is being translated to a driven element in contact with the distal end of the instrument. Similarly, the sensor on an instrument can detect compression at the instrument, while that compression is being translated to an external component, e.g., a driven element.

In additional implementations, one or more device components described herein, e.g., driving elements in implants, can be communicatively coupled with a navigation system that is configured to detect a position of the instrument(s). In one example, the control unit (e.g., ERC) can include or otherwise communicate with a navigation system in order to provide navigation information about a position of instruments. For example, the navigation system can include an optical tracking system such as a camera or laser-based tracking system, a Global Positioning System (GPS), an inertial measurement unit (IMU), an ultrasound based measurement system, other kinds of position systems, or combinations thereof. In certain cases, the navigation system is configured to determine a distance moved by the instrument when the instrument changes position, which the navigation system communicates to the control unit (e.g., for processing by the feedback system). One or more components of a navigation system can be located within or otherwise integrated with a housing that is mounted to or otherwise coupled with one or more of the device components.

In certain cases, a feedback system, or functions thereof, can be integrated into a control unit and/or a controller as described herein. In particular cases, the feedback system is part of a software application and is configured to determine what, if any, force adjustment should be made at a given implant based on the force feedback. In some examples, the feedback system includes a model that correlates force response and force applied during adjustment of the length of an implant. The model can be based at least in part on historical data from a set of implants in distinct bone fixation devices, e.g., similar to implant(s) described herein. According to various implementations, the model can be updated periodically, or on a continuous basis, to provide additional data about force response as compared to force applied in one or more implants. In certain cases, a version of the model can be downloaded or otherwise stored locally at one or more control units and/or controllers and periodically updated, e.g., via a cloud-based or other network-based software update. This approach can reduce the computational and/or storage requirements at control unit(s) and controller(s) that may be local to the implant(s).

In additional implementations, the feedback system is configured to provide post-operative data, post-adjustment data, and analysis of alignment procedure and/or device usage, e.g., to enhance future procedures and/or diagnose inefficiencies in a past procedure. In certain implementations, the feedback system is configured to update the control instructions for control unit(s) based on identified inefficiencies or errors in adjustment quantities (e.g., lengthening, rotation) and/or device usage during/after a given procedure. In particular implementations, the feedback system includes a logic engine configured to modify instructions iteratively, e.g., on a procedure-by-procedure or patient-by-patient basis.

Various additional aspects of the disclosure can include a method of intermedullary adjustment of a patient's bone using the implant(s) described herein. Using FIGS. 1 and 2 strictly for the simplicity of illustration, the method can include adjusting a patient's bone using an intermedullary implant such as implant 10 by: (i) coupling the implant 10 to the patient's bone (e.g., via bone screws or other fasteners at holes 60, 70); and (ii) actuating adjustment of the length of the implant 10 and/or actuating rotation of the implant 10 with an external control device (e.g., ERC or other remote controller). The adjusting of the length can occur before, during, or after adjusting of the rotation. In certain cases, after adjusting the patient's bone, a method can further include: (iii) decoupling the implant from the patient's bone (e.g., via bone screws or other fasteners at holes 60, 70).

In certain cases, a method can include imaging a bone connected with the implant(s) described and illustrated herein. For example, a method can include: (I) coupling or decoupling an implant (e.g., implant 10) with a patient's bone, and (II) imaging the bone with MM and/or X-ray imaging after the coupling or decoupling. After imaging, the method can further include: (III) either (a) adjusting an already coupled implant (e.g., implant 10) or (b) decoupling the already coupled implant (e.g., implant 10) based on feedback from the imaging process.

As noted herein, the implants and associated methods described herein enable effective feedback on bone adjustment procedures, which can reduce treatment times and complications associated with bone adjustment procedures. The various disclosed implementations can improve patient outcomes when compared with conventional implants, for example, enabling more aggressive adjustment profiles (when warranted), increasing adaptability in adjusting bone positioning, and/or enhancing both intraoperative and postoperative engagement with the device. When compared with conventional approaches, the load-sensing implants described according to various implementations provide an efficient mechanism for bone adjustment and feedback on adjustment procedures. The implants described according to various implementations can also reduce health risks for patients when compared with conventional approaches, for example, allowing an implant to provide adjustment feedback with less exposure to imaging.

The functionality described herein, or portions thereof, and its various modifications (hereinafter “the functions”) can be implemented, at least in part, via a computer program product, e.g., a computer program tangibly embodied in an information carrier, such as one or more non-transitory machine-readable media, for execution by, or to control the operation of, one or more data processing apparatus, e.g., a programmable processor, a computer, multiple computers, and/or programmable logic components.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a network.

Actions associated with implementing all or part of the functions can be performed by one or more programmable processors executing one or more computer programs to perform the functions of the calibration process. All or part of the functions can be implemented as, special purpose logic circuitry, e.g., an FPGA and/or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. Components of a computer include a processor for executing instructions and one or more memory devices for storing instructions and data.

In various implementations, components described as being “coupled” to one another can be joined along one or more interfaces. In some implementations, these interfaces can include junctions between distinct components, and in other cases, these interfaces can include a solidly and/or integrally formed interconnection. That is, in some cases, components that are “coupled” to one another can be simultaneously formed to define a single continuous member. However, in other implementations, these coupled components can be formed as separate members and be subsequently joined through known processes (e.g., soldering, fastening, ultrasonic welding, bonding). In various implementations, electronic components described as being “coupled” can be linked via conventional hard-wired and/or wireless means such that these electronic components can communicate data with one another. Additionally, sub-components within a given component can be considered to be linked via conventional pathways, which may not necessarily be illustrated.

Commonly labeled components in the FIGURES are considered to be substantially equivalent components for the purposes of illustration, and redundant discussion of those components is omitted for clarity.

While inventive features described herein have been described in terms of preferred embodiments for achieving the objectives, it will be appreciated by those skilled in the art that variations may be accomplished in view of these teachings without deviating from the spirit or scope of the invention. Also, while this invention has been described according to a preferred use in spinal applications, it will be appreciated that it may be applied to various other uses desiring surgical fixation, for example, the fixation of long bones.

Various example embodiments of devices (e.g., implants) and techniques for moving bone in a patient's body are described herein. In the interest of clarity, not all features of an actual implementation are necessarily described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure. The implants and related systems, program products and methods described herein boast a variety of inventive features and components that warrant patent protection, both individually and in combination.

It is to be understood that any given elements of the disclosed embodiments of the invention may be embodied in a single structure, a single step, a single substance, or the like. Similarly, a given element of the disclosed embodiment may be embodied in multiple structures, steps, substances, or the like.

A number of implementations have been described. Nevertheless, it will be understood that additional modifications may be made without departing from the scope of the inventive concepts described herein, and, accordingly, other implementations are within the scope of the following claims.

1. An implant for moving bone in a patient's body, the implant comprising:
an implantable biocompatible housing;
a first adjustment rod at least partially overlapping the implantable biocompatible housing;
a driver configured to drive the first adjustment rod to enable movement of the first adjustment rod relative to the implantable biocompatible housing; and
a load sensor positioned within the implantable biocompatible housing and configured to indicate a load applied by the driver on the first adjustment rod, wherein the implantable biocompatible housing has a non-circular cross-section that limits rotation of the load sensor.

2. The implant of claim 1, wherein the implant is configured to be wirelessly communicatively coupled to an external control device for transmitting data from the load sensor, wherein the implant comprises a wireless transmitter and one or more processors configured to cause the wireless transmitter to transmit data from the load sensor.

3. (canceled)

4. The implant of claim 1,
wherein the implantable biocompatible housing comprises a first cavity defining an opening;
wherein the first adjustment rod is at least partially disposed within the first cavity;
wherein the driver is positioned between the load sensor and the opening.

5. The implant of claim 1, wherein the driver includes a magnetic actuator configured to be actuated by a magnetic field external to the patient's body, wherein the implant has a proximal end and a distal end, wherein the driver further includes a driven gear system 120 coupled with the magnetic actuator, wherein the load sensor is proximal to the driver, and wherein the driven gear system is distal to the load sensor.

6. (canceled)

7. The implant of claim 1, wherein the load sensor includes a bi-directional load sensor coupled with the driver and configured to indicate both a compressive load and a tensile load applied by the driver to the first adjustment rod, wherein the load sensor is rotationally fixed relative to the implantable biocompatible housing.

8. (canceled)

9. The implant of claim 1, wherein the load sensor includes a button-type load cell.

10. The implant of claim 1, wherein the load sensor is configured to monitor the load applied by the driver on the first adjustment rod during adjustment of the first adjustment rod.

11. The implant of claim 1, the implant further comprising a coupler holding the load sensor in the portion of the implantable biocompatible housing, wherein the load sensor includes a bi-directional load sensor coupled with the driver and configured to indicate both a compressive load and a tensile load applied by the driver to the first adjustment rod.

12. (canceled)

13. (canceled)

14. The implant of claim 1, wherein a proximal end of the first adjustment rod engages a lead screw and is configured to move with the lead screw.

15. The implant of claim 1, wherein the implant is an intermedullary implant, and wherein the implant is configured to aid in treatment of a limb length discrepancy or a bone defect in the patient's body.

16. (canceled)

17. A method of intermedullary adjustment of a patient's bone using comprising inserting the implant of claim 1 into said bone.

18. An implant for moving bone in a patient's body, the implant comprising:
an implantable biocompatible housing having a first cavity;
a first adjustment rod at least partially contained within the first cavity;
a driver configured to drive the first adjustment rod to enable movement relative to the implantable biocompatible housing, wherein the driver includes:
a magnetic actuator configured to be actuated by a magnetic field external to the patient's body; and
a driven gear system coupled with the magnetic actuator; and
a load sensor positioned between the driven gear system of the driver and the first adjustment rod, the load sensor for indicating a load applied by the driver on the first adjustment rod, wherein the implantable biocompatible housing has a non-circular cross-section that limits rotation of the load sensor.

19. The implant of claim 18, wherein the load cell is configured to rotate with the driver relative to the implantable biocompatible housing.

20. The implant of claim 18, wherein the load sensor includes a bi-directional load sensor coupled with the driver and configured to indicate both a compressive load and a tensile load applied by the driver to the first adjustment rod.

21. The implant of claim 18, wherein a proximal end of the first adjustment rod engages a lead screw and is configured to move with the lead screw.

22. The implant of claim 18, wherein the implant is configured for intermedullary placement in a patient, and wherein the implant is configured to aid in treatment of a limb length discrepancy or a bone defect in the patient's body.

23. (canceled)

24. A method of intermedullary adjustment of a patient's bone using the implant of claim 18.

25. A method comprising:
causing an implanted distraction device to expand using an external control device_, wherein the implanted distraction device includes:
an implantable biocompatible housing and a load sensor positioned within the implantable biocompatible housing, wherein the implantable biocompatible housing has a non-circular cross-section that limits rotation of the load sensor;
measuring load sensor data with a load sensor of the implanted distraction device; and
determining, using the load sensor data, an amount of movement of bone in the patient's body.

26. The method of claim 25, wherein the load sensor enables determination of the amount of movement of bone in the patient's body without imaging of the bone in the patient's body, and
wherein the external control device is located proximate to the patient's body during expansion of the implanted distraction device, or remote relative to the patient's body during expansion of the implanted distraction device.

27. (canceled)

28. (canceled)

29. The method of claim 25, wherein determining the amount of movement of the bone in the patient's body includes converting the load sensor data into an estimated bone movement value, wherein the estimated bone movement value compensates for a margin of error.

30. (canceled)
